SEQUENCE LISTING (6 sequences)

SEQ ID NO: 1 - protein, 381 aa, Homo sapiens
MAESHLLQWLLLLLPTLCGPGTAAWTTSSLACAQGPEFWCQSLEQALQCRALGHCLQEVWGHVGADDLCQECEDIVHILNKMAKEAIFQDTMRKFLEQECNVLPLKLLMPQCNQVLDDYFPLVIDYFQNQTDSNGICMHLGLCKSRQPEPEQEPGMSDPLPKPLRDPLPDPLLDKLVLPVLPGALQARPGPHTQDLSEQQFPIPLPYCWLCRALIKRIQAMIPKGALAVAVAQVCRVVPLVAGGICQCLAERYSVILLDTLLGRMLPQLVCRLVLRCSMDDSAGPRSPTGEWLPRDSECHLCMSVTTQAGNSSEQAIPQAMLQACVGSWLDREKCKQFVEQHTPQLLTLVPRGWDAHTTCQALGVCGTMSSPLQCIHSPDL

SEQ ID NO: 2 - protein, 102 aa, Homo sapiens
  MISC_FEATURE: partial sequence of proSP-B (SEQ ID NO 1)
DDSAGPRSPTGEWLPRDSECHLCMSVTTQAGNSSEQAIPQAMLQACVGSWLDREKCKQFVEQHTPQLLTLVPRGWDAHTTCQALGVCGTMSSPLQCIHSPDL

SEQ ID NO: 3 - protein, 50 aa, Homo sapiens
  MISC_FEATURE: partial sequence of proSP-B (SEQ ID NO 1)
PRSPTGEWLPRDSECHLCMSVTTQAGNSSEQAIPQAMLQACVGSWLDREK

SEQ ID NO: 4 - protein, 10 aa, Homo sapiens
  MISC_FEATURE: partial sequence of proSP-B (SEQ ID NO 1)
PRSPTGEWLP

SEQ ID NO: 5 - protein, 12 aa, Homo sapiens
  MISC_FEATURE: partial sequence of proSP-B (SEQ ID NO 1)
QACVGSWLDREK

SEQ ID NO: 6 - protein, 10 aa, Homo sapiens
  MISC_FEATURE: partial sequence of proSP-B (SEQ ID NO 1)
LPKPLRDPLP

(19) United States
(12) Patent Application Publication
(10) Pub. No.: US 2014/0017815 A1
(43) Pub. Date: Jan. 16, 2014

(54) MEASUREMENT OF C-TERMINAL proSP-B

(71) Applicants: Volker Klemt, Weilheim (DE); Christine Boehm, Huenenberg (CH); Andreas Gallusser, Penzberg (DE); Ralf Roeddiger, Gorxheimertal (DE)

(72) Inventors: Volker Klemt, Weilheim (DE); Christine Boehm, Huenenberg (CH); Andreas Gallusser, Penzberg (DE); Ralf Roeddiger, Gorxheimertal (DE)

(73) Assignee: ROCHE DIAGNOSTICS OPERATIONS, INC., Indianapolis, IN (US)

(21) Appl. No.: 14/032,713

(22) Filed: Sep. 20, 2013

Related U.S. Application Data
(63) Continuation of application No. PCT/EP2012/055124, filed on Mar. 22, 2012.

(30) Foreign Application Priority Data
Mar. 25, 2011 (EP) 11159724.1

(51) Int. Cl. G01N 33/68

(52) U.S. Cl. CPC G01N 33/6893
USPC 436/501

(57) ABSTRACT

In vitro methods for obtaining an indication of damage in the broncheoalveolar compartment of the lung comprising, measuring C-terminal proSP-B in a bodily fluid sample and comparing the level measured to a reference level of C-terminal proSP-B, wherein an increased level of C-terminal proSP-B is indicative of damage in the broncheoalveolar compartment of the lung.

### CROSS-REFERENCE TO RELATED APPLICATIONS

This application is a continuation of International Application No. PCT/EP2012/055124 filed Mar. 22, 2012, which claims the benefit of European Patent Application No. 11159724.1 filed Mar. 25, 2011, the disclosures of which are hereby incorporated by reference in their entirety.

### SEQUENCE LISTING

The instant application contains a Sequence Listing which has been submitted in ASCII format via EFS-Web and is hereby incorporated by reference in its entirety. Said ASCII copy, created on Sep. 19, 2013, is named SEQUENCE_LISTING_{—}27378US.txt, and is five thousand nine hundred and forty-five bytes in size.

### BACKGROUND

Breathing is dependent on a gas exchange between air and blood system at the alveoli and its surfactant layer which function as air-blood barrier. The maintenance of alveolar structure and lung surfactant is essential for normal inspiration and expiration cycles. The inherent tendency of the alveolus to collapse at the end of expiration is due to high surface tension generated by an aqueous layer lining the alveolar epithelium. The alveolar stability is achieved by maintenance of a pulmonary surfactant film at the air-liquid interface that reduces surface tension as alveolar surface area decreases. In the absence of surfactant, the collapse of multiple alveoli rapidly progresses to severe respiratory distress, a condition leading to increased alveolo-capillary permeability and the need for ventilatory support.

There are four surfactant proteins, the hydrophilic water soluble surfactant proteins A and D (SP-A and SP-D) which are important for host defense, but have less impact than the hydrophobic surfactant proteins (B and C) on the biophysical. Hydrophobic surfactant proteins B and C (SP-B and SP-C) are important for optimizing surface tension reduction.

### SUMMARY OF THE DISCLOSURE

The present disclosure relates to in vitro methods for obtaining an indication of damage in the broncheoalveolar compartment of the lung. Methods comprise measuring the level of C-terminal proSP-B in a bodily fluid sample and comparing the level measured to a reference level of C-terminal proSP-B, wherein an increased level of C-terminal proSP-B is indicative of damage in the broncheoalveolar compartment of the lung.

In one embodiment the present disclosure relates to an in vitro method for obtaining an indication of a damage in the broncheoalveolar compartment of the lung the method comprising the steps of (a) measuring the level of C-terminal proSP-B in a bodily fluid sample, and (b) comparing the level measured in (a) to a reference level of C-terminal proSP-B, wherein an increased level of C-terminal proSP-B is indicative of a damage in the broncheoalveolar compartment of the lung. In some embodiments, exemplary sequence regions and antibodies thereto are disclosed.

Some embodiments of the present disclosure also provide for a kit comprising at least two antibodies reactive with at least two non-overlapping epitopes comprised in the C-terminal proSP-B sequence in between positions 285 to 334 of C-terminal proSP-B (SEQ ID NO:3).

Additional aspects and advantages of the present disclosure will be apparent in view of the detailed description which follows. It should be understood, however, that the detailed description and the specific examples, while describing exemplary embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The features of this disclosure, and the manner of attaining them, will become more apparent and the disclosure itself will be better understood by reference to the following description of embodiments of the disclosure taken in conjunction with the accompanying drawing.

FIG. 1 is a schematic illustration of a sandwich assay embodiment for measuring proSP-B according to the instant disclosure. The monoclonal antibodies used (clone 1.14.133 and 1.7.41, respectively) and their corresponding binding sites are also indicated in this schematic.

FIG. 2 is a schematic illustration of a sandwich assay embodiment for measuring proSP-B according to the instant disclosure C-terminal proSP-B. The monoclonal antibodies used (clone 1.3.9 and 1.7.41, respectively) and their corresponding binding sites are also indicated in this schematic.

FIG. 3 is a graph of the concentrations of C-proSP-B from Example 4 (dots represent single samples, boxes represent 25% to 75% percentiles, long horizontal lines represent Medians).

Although the drawings represent embodiments of the present disclosure, the drawings are not necessarily to scale and certain features may be exaggerated in order to better illustrate and explain the present disclosure. The exemplifications set out herein illustrate an exemplary embodiment of the disclosure, in one form, and such exemplifications are not to be construed as limiting the scope of the disclosure in any manner.

### BRIEF DESCRIPTION OF THE SEQUENCE LISTING

SEQ ID NO.1: is the proSP-B sequence corresponds to amino acids 1 to 381 of the proSP-B (given as “ID=PSPB_HUMAN; reviewed; 381 AA; AC P07988; Q96R04; as integrated into UniProtKB/Swiss-Prot data bank).

SEQ ID NO.2: is a partial sequence of proSP-B, spanning from amino acids 280 to 381 of proSP-B.

SEQ ID NO.3: is a partial sequence of proSP-B, spanning from amino acids 285 to 334 of proSP-B.

SEQ ID NO.4: is a partial sequence of proSP-B, spanning from amino acids 285 to 294 of proSP-B.

SEQ ID NO.5: is a partial sequence of proSP-B, spanning from amino acids 323 to 334 of proSP-B.

SEQ ID NO.6: is a partial sequence of proSP-B, spanning from amino acids 160 to 169 of proSP-B.

Although the sequence listing represents an embodiment of the present disclosure, the sequence listing is not to be construed as limiting the scope of the disclosure in any manner and may be modified in any manner as consistent with the instant disclosure and as set forth herein.

### DETAILED DESCRIPTION OF THE DISCLOSURE

The embodiments disclosed herein are not intended to be exhaustive or limit the disclosure to the precise form disclosed in the following detailed description. Rather, the embodiments are chosen and described so that others skilled in the art may utilize their teachings.

The present disclosure relates to an in vitro method for obtaining an indication of damage in the broncheoalveolar compartment of the lung. This method comprises measuring the level of proSP-B, for example C-terminal proSP-B, in a bodily fluid sample and comparing the level measured to a reference level of proSP-B, for example C-terminal proSP-B, wherein an increased level of the proSP-B, for example C-terminal proSP-B is indicative of damage in the broncheoalveolar compartment of the lung.

Surfactant Protein B (SP-B) is a 79-amino acid peptide, produced by the proteolytic cleavage of SP-B proprotein (proSP-B; SEQ ID NO: 1) that is processed to a smaller, lipid-associated peptide in the distal secretory pathway within the type II cell. Processing of proSP-B occurs in the multivesicular body (MVB) and lamellar body (LB) compartments.

SP-B facilitates the stability and rapid spreading of surfactant phospholipids during respiratory cycles. It maintains the molecular continuity of the monolayer of the lipid and peptide at the air-water interface during breathing and facilitates the incorporation of lipid from the lung aqueous subphase into the lipid monolayer at the alveolar air-water interface. A lack of SP-B shortly after birth can be lethal. The fully processed mature peptide facilitates organization of surfactant membranes in the lamellar body, possibly through its ability to promote membrane-membrane contacts, perturbation of lipid packing, and membrane fusion. SP-B is also expressed in nonciliated bronchiolar Clara cells, although the role of SP-B in the Clara cell is not known.

Processing of the SP-B preproprotein to its mature peptide occurs during transit through the secretory pathway from the endoplasmic reticulum to the Golgi network and the multivesicular bodies in the type II epithelial cell. Entry of the SP-B preproprotein into the secretory pathway is mediated by the N-terminal signal peptide which is cleaved upon translation of the proprotein into the endoplasmic reticulum. Transit of SP-B out of the endoplasmic reticulum is dependent on the N-terminal propeptide, which possibly facilitates folding and/or sequestration of the hydrophobic mature peptide. The C-terminal propeptide, (amino acids 280 to 381 of proSP-B=SEQ ID NO: 2) however, seems not to be required for sorting of SP-B to secretory granules.

The role of the C-terminal propeptide of SP-B has been described as not well-known or as possibly irrelevant. For example, Weaver et al. (1998) conclude that C-terminal proSP-B does appear “not to be required for sorting of SP-B to secretory granules”. Akinbi, H. et al., J. Biol. Chem. 272 (1997) 9640-9647, have investigated the role of the C-terminal propeptide and concluded from their results “that the 102-residue C-terminal propeptide of SP-B is not required for the normal structure and function of extracellular surfactant. Further, a different role for pro-SP-B (as compared to the mature SP-B protein) has been postulated by Pryhuber (Pryhuber, G. S., Mol. Gen. Metabol. 64 (1998) 217-228) due to different timely expression of mature and SP-B proprotein in fetal lung. In humans, the SP-B mRNA and proprotein are detected as early as 14-15 weeks of gestation prior to detection of mature surfactant. The early appearance of SP-B proprotein in fetal lung suggests that the proprotein may have a function in early development which is not directly related to surface tension reduction, e.g. in an adult. Also, proSP-B has been found in the tracheal aspirates from newborn infants (100 out of 101) but not on the broncheoalveolar lavage of adults (0 out of 6) (Havmas, A. et al., Neonatology 95 (2009) 117-124).

Despite questions around the biological function of C-terminal proSP-B, especially in adults, and despite the absence of proSP-B from the broncheoalveolar lavage of adults, the inventors of the present disclosure have surprisingly found that measurement of C-terminal proSP-B is valuable in obtaining an indication of a damage in the broncheoalveolar compartment of the lung. The inventors of the present disclosure have surprisingly been able to demonstrate that C-terminal proSP-B is useful in the assessment of a damage in the broncheoalveolar compartment of the lung, where increased concentrations of protein C-terminal proSP-B in a sample as compared to normal controls have been found to be indicative of a damage in the broncheoalveolar compartment of the lung.

In some embodiments, the present disclosure relates to an in vitro method for diagnosing a damage in the broncheoalveolar compartment of the lung the method comprising the steps of (a) measuring the level of C-terminal proSP-B in a bodily fluid sample, and (b) comparing the level measured in (a) to a reference level of C-terminal proSP-B, wherein an increased level of C-terminal proSP-B is indicative of a damage in the broncheoalveolar compartment of the lung.

C-terminal proSP-B, as used herein, relates to proSP-B and all cleavage products or fragments thereof comprising the C-terminal sequence of proSP-B as defined in SEQ ID NO: 3. Measurement of C-terminal proSP-B thus relates to the measurement of proSP-B and those fragments or cleavage products thereof comprising SEQ ID NO:3. C-terminal proSP-B includes, but is not limited to, the following: proSP-B (i.e. the proSP-B of SEQ ID NO:1, comprising the N-terminal propeptide sequence the sequence stretch representing the mature SP-B and the C-terminal pro SP-B); the mid-molecular plus C-terminal fragment (i.e. the amino acids from position 201 to 381 of SEQ ID NO:1) and the C-terminal proSP-B fragment (i.e. the amino acids from position 280 to 381 of SEQ ID NO:1). As obvious to the artisan, proSP-B and fragments comprising the C-terminal proSP-B sequence of SEQ ID NO: 3 may be present as monomers and/or dimers, respectively.

As set forth herein, the inventors of the present disclosure have, for the first time, found and can establish that an increased concentration of C-terminal proSP-B in a body fluid is indicative for a damage in the broncheoalveolar compartment of the lung. According to at least some embodiments of the present disclosure, practicing of the instant disclosure will employ, unless otherwise indicated, conventional techniques of molecular biology (including recombinant techniques), microbiology, cell biology, biochemistry, and immunology, which are within the skill of the art. Such techniques are explained in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. 1. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994).

Unless defined otherwise, technical and scientific terms used herein have the same meaning as commonly understood by one of ordinary skill in the art to which this disclosure belongs. Singleton et al., Dictionary of Microbiology and Molecular Biology, 2nd ed., J. Wiley & Sons, New York, N.Y. (1994); March, Advanced Organic Chemistry Reactions, Mechanisms and Structure 4th ed., John Wiley & Sons, New York, N.Y. (1992); Lewin, B., Genes V, published by Oxford University Press (1994), ISBN 0-19-854287 9); Kendrew, J. et al. (eds.), The Encyclopedia of Molecular Biology, published by Blackwell Science Ltd. (1994), ISBN 0-632-02182-9); and Meyers, R. A. (ed.), Molecular Biology and Biotechnology: a Comprehensive Desk Reference, published by VCH Publishers, Inc. (1995), ISBN 1-56081-569 8) provide one skilled in the art with a general guide to many of the terms used in the present application.

As used herein, each of the following terms has the meaning associated with it in this section.

The articles “a” and “an” are used herein to refer to one or to more than one (i.e. to at least one) of the grammatical object of the article. By way of example, “a marker” means one marker or more than one marker. The term “at least” is used to indicate that optionally one or more than one further objects may be present.

The expression “one or more” denotes 1 to 50, for example 1 to 20 and/or also 2, 3, 4, 5, 6, 7, 8, 9, 10, 12, or 15.

The term “marker” or “biochemical marker” as used herein refers to a molecule to be used as a target for analyzing an individual's test sample. In one embodiment examples of such molecular targets are proteins or polypeptides. Proteins or polypeptides used as a marker in the present disclosure are contemplated to include naturally occurring variants of said protein as well as fragments of said protein or said variant, in particular, immunologically detectable fragments. Immunologically detectable fragments may comprise at least 6, 7, 8, 10, 12, 15 or 20 contiguous amino acids of said marker polypeptide. One of skill in the art would recognize that proteins which are released by cells or present in the extracellular matrix may be damaged, e.g., during inflammation, and could become degraded or cleaved into such fragments. Certain markers are synthesized in an inactive form, which may be subsequently activated by proteolysis. As the skilled artisan will appreciate, proteins or fragments thereof may also be present as part of a complex. Such complex also may be used as a marker in the sense of the present disclosure. In addition, or in the alternative a marker polypeptide or a variant thereof may carry a post-translational modification. Exemplary posttranslational modifications include glycosylation, acylation, or phosphorylation.

The term “diagnosing” as used herein means assessing whether in a subject a damage in the broncheoalveolar compartment of the lung is present, or not. As will be understood by those skilled in the art, such an assessment is usually not intended to be correct for 100% of the subjects to be diagnosed. The term, however, requires that the assessment of the presence or absence of the damage is correct for a statistically significant portion of the subjects (e.g. a cohort in a cohort study). Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Exemplary confidence intervals include at least 90%, at least 95%, at least 97%, at least 98% or at least 99%. The p-values are, for example, 0.1, 0.05, 0.01, 0.005, or 0.0001. It follows from the above that in a specific embodiment of the disclosure diagnosing means obtaining an indication of a damage in the broncheoalveolar compartment of the lung. The phrase “obtaining an indication of a damage in the broncheoalveolar compartment of the lung” is used to make clear that the method according to the present disclosure will alone or together with other markers or variables be indicative of a damage in the broncheoalveolar compartment of the lung, e.g., aid the physician in assessing a damage in the broncheoalveolar compartment of the lung. The method will e.g. be useful to establish or confirm the absence or presence of a damage in the broncheoalveolar compartment of the lung.

A “damage” in the broncheoalveolar compartment of the lung is present, whenever a disturbance of the modulation of surface tension at the alveolar air-liquid interface occurs. A damage in the broncheoalveolar compartment of the lung comprises, e.g., an injury to and/or a dysfunction of the broncheoalveolar membrane as well as repair processes at the broncheoalveolar membrane. In an embodiment, the damage in the broncheoalveolar compartment of the lung is caused by a disease, by a disorder or by a behavior selected from the group consisting of: Smoking, chronic obstructive pulmonary disease (COPD), pneumonia, pneumoconiosis, non-small-cell lung carcinoma, bronchitis, adenocarcinoma of the lung (Adeno-Ca), small-cell lung carcinoma (SCC), and interstitial lung disease, like, e.g., asbestosis, silikosis, idiopathic pulmonary fibrosis (IPF), or sarcoidosis.

A “marker indicative of a damage in the broncheoalveolar compartment of the lung” in the sense of the present disclosure is a marker that, as single marker, or if combined with the marker C-terminal proSP-B, adds relevant information in the assessment of a damage in the broncheoalveolar compartment of the lung. The information is considered relevant or of additive value if at a given specificity the sensitivity, or if at a given sensitivity the specificity, respectively, for the assessment of a damage in the broncheoalveolar compartment of the lung can be improved by including said marker into a marker panel (marker combination) already comprising the marker C-terminal proSP-B. According to at least some embodiments, the improvement in sensitivity or specificity, respectively, is statistically significant at a level of significance of p=0.05, 0.02, 0.01 or lower.

The term “sample” or “test sample” as used herein refers to a bodily fluid sample obtained from an individual for the purpose of evaluation in vitro. Exemplary samples are body fluids such as broncheoalveolar lavage, sputum, serum, plasma, or whole blood. In one embodiment the bodily fluid sample is selected from broncheoalveolar lavage, serum or plasma. In one embodiment the bodily fluid is serum or plasma. In one embodiment the method according to the present disclosure is practiced with serum as liquid sample material. In one embodiment the method according to the present disclosure is practiced with plasma as liquid sample material.

The expression “comparing the level measured to a reference level . . . ” is merely used to further illustrate what is obvious to the skilled artisan anyway. A reference level is established in a control sample. The control sample may be an internal or an external control sample. In one embodiment an internal control sample is used, i.e. the marker level(s) is(are) assessed in the test sample as well as in one or more other sample(s) taken from the same subject to determine if there are any changes in the level(s) of said marker(s). This may for example be useful in assessing the efficacy of therapy. In another embodiment an external control sample is used. For an external control sample the presence or amount of a marker in a sample derived from the individual is compared to its presence or amount in an individual known to suffer from, or known to be at risk of, a given condition; or an individual known to be free of a given condition, i.e., a “normal individual”. For example, a marker level in a patient sample can be compared to a level known to be associated with a specific course of disease. Usually the sample's marker level is directly or indirectly correlated with a diagnosis of a disease or of a certain physiological or pathological status. The marker level is e.g. used to determine whether an individual is at risk of a disease. Alternatively, the sample's marker level can e.g. be compared to a marker level known to be associated with a response to therapy, the diagnosis of a disease or of a certain physiological or pathological status, the guidance for selecting an appropriate therapy, in judging the risk of disease progression, or in the follow-up of patients. Depending on the intended diagnostic use an appropriate control sample is chosen and a control or reference value for the marker established therein. It will be appreciated by the skilled artisan that such control sample in one embodiment is obtained from a reference population that is age-matched and free of confounding diseases. As also clear to the skilled artisan, the absolute marker values established in a control sample will be dependent on the assay used. According to some embodiments, samples from 100 well-characterized individuals from the appropriate reference population are used to establish a control (reference) value. Also the reference population may be chosen to consist of 20, 30, 50, 200, 500 or 1000 individuals. Healthy individuals represent an exemplary reference population for establishing a control value.

The term “measurement”, “measuring” or “determining” comprises a qualitative, semi-quantitative or a quantitative measurement. In the present disclosure C-terminal proSP-B, i.e. those proSP-B fragments comprising the C-terminal sequence of proSP-B as defined in SEQ ID NO:3, is measured in a bodily fluid sample. In an exemplary embodiment the measurement is a semi-quantitative measurement, i.e. it is determined whether the concentration of C-terminal proSP-B is above or below a cut-off value. As the skilled artisan will appreciate, in a Yes-(presence) or No-(absence) assay, the assay sensitivity is usually set to match the cut-off value.

The values for protein C-terminal proSP-B as determined in a control group or a control population are for example used to establish a cut-off value or a reference range. A value above such cut-off value or out-side the reference range at its higher end is considered as elevated or as indicative of a damage in the broncheoalveolar compartment of the lung. In some embodiments a fixed cut-off value is established. Such cut-off value is chosen to match the diagnostic question of interest.

In some embodiments the cut-off is set to result in a specificity of 90%, and in some embodiments the cut-off is set to result in a specificity of 95%, or even set to result in a specificity of 98%.

In some embodiments the cut-off is set to result in a sensitivity of 90%, or in some embodiments the cut-off is set to result in a sensitivity of 95%, or even set to result in a sensitivity of 98%.

In some embodiments values for C-terminal proSP-B as determined in a control group or a control population are used to establish a reference range. In some embodiments an concentration of C-terminal proSP-B is considered as elevated if the value determined is above the 90%-percentile of the reference range. In further embodiments a concentration of C-terminal proSP-B is considered as elevated if the value determined is above the 95%-percentile, the 96%-percentile, the 97%-percentile or the 97.5%-percentile of the reference range.

A value above the cut-off value can for example be indicative for the presence of a damage in the broncheoalveolar compartment of the lung. A value below the cut-off value can for example be indicative for the absence of a damage in the broncheoalveolar compartment of the lung.

In further embodiments, the measurement of C-terminal proSP-B is a quantitative measurement. In some embodiments the concentration of protein C-terminal proSP-B is correlated to an underlying diagnostic question.

As the skilled artisan will appreciate, any such measurement is made in vitro. The sample (test sample) is discarded afterwards. The sample is solely used for the in vitro diagnostic method of the disclosure and the material of the sample is not transferred back into the patient's body. Typically, the sample is a bodily fluid sample.

The method according to the present disclosure is based on a liquid or bodily fluid sample which is obtained from an individual and on the in vitro determination of C-terminal proSP-B in such sample. An “individual” as used herein refers to a single human or non-human organism. Thus, the methods and compositions described herein are applicable to both human and veterinary disease.

According to at least some embodiments of the instant disclosure, the C-terminal proSP-B is specifically measured or determined in vitro from a liquid sample by use of at least one specific binding agent to C-terminal proSP-B. According to at least some illustrative embodiments of the instant disclosure, a specific binding agent may comprise an antibody reactive with SEQ ID NO: 3. In some embodiments according to the present disclosure, the concentration of C-terminal proSP-B is determined. In some embodiments, the concentration of C-terminal proSP-B is determined in vitro from a bodily fluid sample by use of a specific binding agent. A specific binding agent, according to the instant disclosure may be an antibody, or an antigen-binding fragment thereof, to C-terminal proSP-B. A specific binding agent has at least an affinity of 10⁷ l/mol for its corresponding target molecule. The specific binding agent may have an affinity of 10⁸ l/mol or even of 10⁹ l/mol for its target molecule.

As the skilled artisan will appreciate the term specific is used to indicate that other biomolecules present in the sample do not significantly bind to the binding agent used in the detection of the C-terminal proSP-B sequence of SEQ ID NO: 3. According to the instant disclosure, the level of binding to a biomolecule other than the target molecule may result in a binding affinity which is at most only 10% or less, only 5% or less only 2% or less or only 1% or less of the affinity to the target molecule, respectively. An exemplary specific binding agent, according to at least some embodiments, fulfils both the above minimum criteria for affinity as well as for specificity.

Examples of specific binding agents include peptides, peptide mimetics, aptamers, spiegelmers, darpins, ankyrin repeat proteins, Kunitz type domains, antibodies, single domain antibodies, (see: Hey, T. and Fiedler, E., et al., Trends Biotechnol. 23 (2005) 514-522) and monovalent fragments of antibodies. In certain embodiments, the specific binding agent is a polypeptide. In some embodiments the specific binding agent is an antibody or a monovalent antibody fragment, for example a monovalent fragment derived from a monoclonal antibody. Monovalent antibody fragments include, but are not limited to Fab, Fab′-SH, single domain antibody, Fv, and scFv fragments, as provided below.

The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

“Native antibodies” are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues are believed to form an interface between the light-chain and heavy-chain variable domains.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The subunit structures and three-dimensional configurations of different classes of immunoglobulins are well known and described generally in, for example, Abbas et al., Cellular and Mol. Immunology, 4th ed., W.B. Saunders, Co. (2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody, for example comprising the antigen-binding region thereof. Examples of antibody fragments include Fab, Fab′, F(ab′)2, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody-hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of an antibody, wherein these domains are present in a single polypeptide chain. Generally, the scFv polypeptide further comprises a polypeptide linker between the VH and VL domains that enables the scFv to form the desired structure for antigen binding. For a review of scFv, see, e.g., Plueckthun, In: The Pharmacology of Monoclonal Antibodies, Vol. 113, Rosenburg and Moore (eds.), Springer-Verlag, New York (1994) pp. 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 0 404 097; WO 1993/01161; Hudson et al., Nat. Med. 9 (2003) 129-134; and Hollinger et al., PNAS USA 90 (1993) 6444-6448. Triabodies and tetrabodies are also described in Hudson et al., Nat. Med. 9 (2003) 129-134.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target binding sequence is also a monoclonal antibody of this disclosure. In contrast to polyclonal antibody preparations, which typically include different antibodies directed against different determinants (epitopes), each monoclonal antibody of a monoclonal-antibody preparation is directed against a single determinant on an antigen. In addition to their specificity, monoclonal-antibody preparations are advantageous in that they are typically uncontaminated by other immunoglobulins.

For the achievements as disclosed in the present disclosure antibodies from various sources may be used. Standard protocols for obtaining antibodies can be as well used as modern alternative methods. Alternative methods for generation of antibodies comprise amongst others the use of synthetic or recombinant peptides, representing a clinically relevant epitope of C-terminal proSP-B for immunization. Alternatively, DNA immunization also known as DNA vaccination may be used. Clearly monoclonal antibodies or polyclonal antibodies, respectively, from different species, e.g., rabbits, sheep, goats, rats or guinea pigs can be used. Since monoclonal antibodies can be produced in any amount required with constant properties, they represent exemplary and useful tools in development of an assay for clinical routine.

As the skilled artisan will appreciate now, that C-terminal proSP-B has been identified as a marker which is indicative of a damage in the broncheoalveolar compartment of the lung, various immunodiagnostic procedures may be used to reach data comparable to those shown in the present disclosure.

For determination of C-terminal proSP-B the sample obtained from an individual is incubated in vitro with the specific binding agent for C-terminal proSP-B under conditions appropriate for formation of a binding agent C-terminal proSP-B complex. Such conditions need not be specified, since the skilled artisan without any inventive effort can easily identify such appropriate incubation conditions. The amount of binding agent C-terminal proSP-B complex is determined and used in the assessment of a damage in the broncheoalveolar compartment of the lung. As the skilled artisan will appreciate there are numerous methods to determine the amount of the specific binding agent C-terminal proSP-B complex all described in detail in relevant textbooks (cf., e.g., Tijssen, P., supra, or Diamandis, E. P., and Christopoulos, T. K. (eds.), Immunoassay, Academic Press, Boston (1996)).

Immunoassays are well known to the skilled artisan. Methods for carrying out such assays as well as practical applications and procedures are summarized in related textbooks. Examples of related textbooks are Tijssen, P., Preparation of enzyme-antibody or other enzyme-macromolecule conjugates, In: Practice and theory of enzyme immunoassays, pp. 221-278, Burdon, R. H. and v. Knippenberg, P. H. (eds.), Elsevier, Amsterdam (1990), and various volumes of Methods in Enzymology, Colowick, S. P., and Caplan, N. O. (eds.), Academic Press), dealing with immunological detection methods, especially volumes 70, 73, 74, 84, 92 and 121.

The present disclosure also relates in an embodiment to the use of an antibody specifically binding to C-terminal proSP-B in a method according to the present disclosure. In some embodiments, a method according to the present disclosure C-terminal proSP-B is measured in an immunoassay procedure. In some embodiments, a method according to the present disclosure C-terminal proSP-B is measured in a competitive immunoassay. In some such assay formats, a binding agent specifically binding to C-terminal proSP-B of SEQ ID NO: 2 or SEQ ID NO: 3, respectively, is used. In a mixture labeled C-terminal proSP-B that has been added to the mixture and C-terminal SP-B comprised in a sample compete for binding to the specific binding agent. The extent of such competition can be measured according to standard procedures.

In some embodiments C-terminal proSP-B is measured in a sandwich immunoassay (sandwich-type assay format). In such sandwich-type assay, a first specific binding agent is used to capture C-terminal proSP-B on the one side and a second specific binding agent, which is labelled to be directly or indirectly detectable, is used on the other side of such sandwich. In a sandwich-type assay format at least one antibody specifically binding to C-terminal proSP-B is used. As will be appreciated, it will be often advantageous and represents a illustrative embodiment to use two or more antibodies specifically binding to C-terminal proSP-B in order to set up a sandwich-type immunoassay.

In some embodiments the method according to the present disclosure is based on the measurement of C-terminal proSP-B, wherein said measurement of C-terminal proSP-B is performed in a sandwich immunoassay employing at least two antibodies reactive with at least two non-overlapping epitopes comprised in the C-terminal proSP-B sequence (SEQ ID NO:2—positions 280 to 381).

In some embodiments the method according to the present disclosure is based on the measurement of C-terminal proSP-B, wherein said measuring of C-terminal proSP-B is performed in a sandwich immunoassay employing at least two antibodies reactive with at least two non-overlapping epitopes comprised in the C-terminal proSP-B sequence (SEQ ID NO:3—positions 285 to 334).

The data presented in the frame-work of the present disclosure have been generated by using monoclonal antibodies to C-terminal proSP-B binding to epitopes comprised in well-defined short sequences of C-terminal proSP-B. In some embodiments the present disclosure relates to a method of measuring-terminal proSP-B, wherein a monoclonal antibody is used that reacts with an epitope comprised in the sequence stretch consisting of amino acids 285 to 294 of human proSP-B (SEQ ID NO: 4). In one embodiment the present disclosure relates to a method of measuring-terminal proSP-B, wherein a monoclonal antibody is used that reacts with an epitope comprised in the sequence stretch consisting of amino acids 323 to 334 of human proSP-B (SEQ ID NO: 5).

In one embodiment method according to the present disclosure is based on the measurement of C-terminal proSP-B by a sandwich immuno assay wherein a first monoclonal antibody that reacts with an epitope comprised in the sequence stretch consisting of amino acids 285 to 294 of human proSP-B (SEQ ID NO: 4) and a second monoclonal antibody that reacts with an epitope comprised in the sequence stretch consisting of amino acids 323 to 334 of human proSP-B (SEQ ID NO: 5) is used.

The generation and the use of monoclonal antibodies binding to the C-terminal proSP-B via an epitope comprised in SEQ ID NO: 4 or SEQ ID NO: 5, respectively, represents an embodiment of the present disclosure. The epitope comprised in SEQ ID NO: 4 or SEQ ID NO: 5, respectively, consists of at least four amino acids comprised in the peptide sequences given. In one further embodiment the epitopes consists of at least five amino acids comprised in the peptide sequences of SEQ ID NO: 4 or SEQ ID NO: 5, respectively. In yet one further embodiment the epitopes consists of at least six amino acids comprised in the peptide sequences of SEQ ID NO: 4 or SEQ ID NO: 5, respectively.

The inventors of the present disclosure surprisingly are able to detect C-terminal proSP-B in a body fluid sample. Even more surprising they are able to demonstrate that the presence of C-terminal proSP-B in such liquid sample obtained from an individual can be correlated to damage in the broncheoalveolar compartment of the lung. No tissue and no biopsy sample is required to make use of the marker C-terminal proSP-B. It will be appreciated that a particular and unique benefit of the disclosure is the ease of the in vitro methods of the present disclosure which may be performed requiring only (e.g. a small aliquot of) a simple body fluid sample.

In further exemplary embodiments, the present disclosure relates to use of C-terminal pro-SP-B as a marker molecule to obtain an indication of a damage in the broncheoalveolar compartment of the lung by an in vitro analysis of a liquid sample obtained from an individual.

An exemplary scenario in the diagnostic field is a situation wherein a single event or process causes the respective disease as, e.g., in infectious diseases. As the skilled artisan will appreciate, no biochemical marker is diagnostic with 100% specificity and at the same time 100% sensitivity for a given multifactorial disease, as for example for a damage in the broncheoalveolar compartment of the lung. Rather, biochemical markers are used to obtain an indication with regard to an underlying diagnostic question, e.g., the presence, absence, or the severity of a disease. Therefore in routine clinical diagnosis, generally various clinical symptoms and biological markers are considered together in the assessment of an underlying disease. The skilled artisan is fully familiar with the mathematical/statistical methods that routinely are used to calculate a relative risk or likelihood for the diagnostic question to be assessed. In routine clinical practice various clinical symptoms and biological markers are generally considered together by a physician in the diagnosis, treatment, and management of the underlying disease.

Obtaining an indication of a damage in the broncheoalveolar compartment of the lung by in vitro measurement of C-terminal proSP-B will be of advantage in at least one or more of the following aspects: screening; staging of disease; monitoring of disease progression; prognosis; guidance of therapy and monitoring of the response to therapy. Exemplary areas of diagnostic relevance for obtaining an indication of a damage in the broncheoalveolar compartment of the lung include screening, staging of disease, monitoring of disease progression and monitoring of the response to therapy.

Screening (Assessment Whether Individuals are Likely to have a Damage in the Broncheoalveolar Compartment of the Lung):

Screening is defined as the systematic application of a test to identify individuals with an increased likelihood for the presence of a disease or pathological state, e.g. in the present case of a damage in the broncheoalveolar compartment of the lung. The screening population may be composed of individuals known to be at higher than average risk of a damage in the broncheoalveolar compartment of the lung. For example, a screening population for a damage in the broncheoalveolar compartment of the lung is composed of individuals known to be at higher than average risk of a damage in the broncheoalveolar compartment of the lung.

Screening in the sense of the present disclosure relates to the unbiased assessment of individuals regarding their risk of having a damage in the broncheoalveolar compartment of the lung. In an embodiment the method according to the present disclosure is used for screening purposes. I.e., it is used to assess subjects without a prior diagnosis of a damage in the broncheoalveolar compartment of the lung by a) measuring C-terminal proSP-B in a sample in vitro, and b) comparing the concentration of C-terminal proSP-B measured in step (a) with a reference concentration of C-terminal proSP-B, wherein a concentration of C-terminal proSP-B above the reference concentration is indicative for the presence of a damage in the broncheoalveolar compartment of the lung. In some embodiments, a body fluid sample such as blood, serum, or plasma is used as a sample in the screening for a damage in the broncheoalveolar compartment of the lung.

Measurement of C-terminal proSP-B will aid the physician to assess the presence or absence of a damage in the broncheoalveolar compartment of the lung in an individual suspected to have such damage in the broncheoalveolar compartment of the lung.

In some embodiments the present disclosure relates to an in vitro method of assessing for a subject the presence or absence of a damage in the broncheoalveolar compartment of the lung, the method comprising a) determining the concentration of C-terminal proSP-B in a sample, and b) comparing the concentration of protein C-terminal proSP-B determined in step (a) with a cut-off value for C-terminal proSP-B established in a reference population, wherein a concentration of C-terminal proSP-B above the cut-off value is indicative for the presence of a damage in the broncheoalveolar compartment of the lung. In an embodiment the present disclosure relates to an in vitro method of assessing for a subject the presence or absence of a damage in the broncheoalveolar compartment of the lung, the method comprising a) determining the concentration of C-terminal proSP-B in a sample, and b) comparing the concentration of protein C-terminal proSP-B determined in step (a) with a cut-off value for C-terminal proSP-B established in a reference population, wherein a concentration of C-terminal proSP-B below the cut-off value is indicative for the absence of a damage in the broncheoalveolar compartment of the lung.

Some embodiments of the present disclosure refer to the screening of a population to distinguish between individuals who are probably free from a damage in the broncheoalveolar compartment of the lung and individuals which probably have a damage in the broncheoalveolar compartment of the lung. The latter group of individuals may then be subject to further appropriate diagnostic procedures.

Staging of Patients:

In some embodiments the present disclosure relates to an in vitro method aiding in the staging of a damage in the broncheoalveolar compartment of the lung patients, comprising the steps of a) determining the concentration of C-terminal proSP-B in a sample, b) comparing the concentration of C-terminal proSP-B determined in step (a) with a reference concentration of C-terminal proSP-B, and indicating stage or severity of a damage in the broncheoalveolar compartment of the lung by comparing the concentration determined in step (a) to the concentration of this marker previously established as indicative for the stage or severity of the damage.

In some embodiments the present disclosure relates to an in vitro method a stage of a damage in the broncheoalveolar compartment of the lung, comprising the steps of a) measuring the concentration of C-terminal proSP-B in a body fluid sample, b) comparing the concentration of C-terminal proSP-B determined in step (a) with a reference concentration of C-terminal proSP-B, and staging a damage in the broncheoalveolar compartment of the lung by comparing the concentration determined in step (a) to the concentration of this marker to the reference value(s) indicative of a certain stage of damage in the broncheoalveolar compartment of the lung. In some embodiments, the level of C-terminal proSP-B may be used as an aid in classifying the individuals investigated into the group of individuals that are clinically “normal”, into the group of patients at risk of having a damage in the broncheoalveolar compartment of the lung, and the group of patients having a damage in the broncheoalveolar compartment of the lung. In certain embodiments stages may further be grouped as mild, moderate, severe or very severe, respectively.

Prognosis:

Prognostic indicators can be defined as clinical, pathological or biochemical features of a damage in the broncheoalveolar compartment of the lung patients that predict with certain likelihood the disease outcome. Their main use is to help to rationally plan patient management, i.e. to avoid undertreatment of aggressive disease and overtreatment of indolent disease, respectively.

As the level of C-terminal proSP-B alone significantly contributes to the differentiation of patients having a damage in the broncheoalveolar compartment of the lung patients from healthy controls, it has to be expected that it will aid in assessing the prognosis of patients suffering from a damage in the broncheoalveolar compartment of the lung.

Obtaining an Indication of Disease Progression:

At present it is very difficult to predict with a reasonable likelihood whether a patient diagnosed with a damage in the broncheoalveolar compartment of the lung has a more or less stable status or whether the disease will progress.

Progression of disease, i.e. of a damage in the broncheoalveolar compartment of the lung disease may be evaluated in vitro by monitoring of the concentration of C-terminal proSP-B in test samples, especially by taking one or more consecutive samples. In some embodiments the present disclosure relates to an in vitro method for obtaining an indication of disease progression in a patient suffering from a damage in the broncheoalveolar compartment of the lung the method comprising the steps of a) determining the concentration of C-terminal proSP-B in a sample, b) comparing the concentration of C-terminal proSP-B determined in step (a) with a reference concentration of C-terminal proSP-B, and obtaining an indication of disease progression by comparing the concentration determined in step (a) to the concentration of this marker as determined in a sample taken from the same patient at a previous point in time. As will be appreciated an increase in the level of C-terminal proSP-B over time is indicative of disease progression.

Monitor a Patient's Response to Therapy:

The method according to the present disclosure, when used in patient monitoring, may be used in the follow-up of patients and e.g. help to assess efficacy of a treatment targeted at reducing damage in the broncheoalveolar compartment of the lung.

In some embodiments the present disclosure relates to an in vitro method for monitoring a patient's response to a treatment targeted at reducing damage in the broncheoalveolar compartment of the lung-therapy, comprising the steps of a) determining the concentration of C-terminal proSP-B in a body fluid sample, b) comparing the concentration of C-terminal proSP-B determined in step (a) with a reference concentration of C-terminal proSP-B, and of monitoring a patient's response to a damage in the broncheoalveolar compartment of the lung therapy by comparing the concentration determined in step (a) to the concentration of this marker to its reference value. In an exemplary embodiment the body fluid sample is selected from the group consisting of serum, plasma and whole blood.

Monitoring a patient's response to therapy can be practiced e.g. by establishing the pre- and post-therapeutic marker level for C-terminal proSP-B and by comparing the pre- and the post-therapeutic marker level.

A patient's response to a treatment targeted at reducing damage in the broncheoalveolar compartment of the lung disease may be evaluated in vitro by monitoring the concentration of C-terminal proSP-B in test samples over time. In some embodiments the present disclosure relates to an in vitro method for monitoring a patient's response to a treatment targeted at reducing damage in the broncheoalveolar compartment of the lung, comprising the steps of a) determining the concentration of C-terminal proSP-B in a sample, b) comparing the concentration of C-terminal proSP-B determined in step (a) with a concentration of C-terminal proSP-B established in a previous sample, wherein a decrease in C-terminal proSP-B is indicative of a positive response to said treatment.

The level of C-terminal proSP-B appears to also be appropriate to monitor a patient's response to therapy. The present disclosure thus also relates to the use of C-terminal proSP-B in monitoring a patient's response to therapy, wherein a decreased level of C-terminal proSP-B is a positive indicator for an effective treatment targeted at reducing damage in the broncheoalveolar compartment of the lung.

Marker Combinations:

The present disclosure therefore relates to embodiments of the use of C-terminal proSP-B as one marker of a marker panel for obtaining an indication of a damage in the broncheoalveolar compartment of the lung. Such marker panel comprises C-terminal proSP-B and one or more additional marker for a damage in the broncheoalveolar compartment of the lung. Certain combinations of markers will e.g. be advantageous in the screening for a damage in the broncheoalveolar compartment of the lung.

As the skilled artisan will appreciate there are many ways to use the measurements of two or more markers in order to improve the diagnostic question under investigation.

Biochemical markers can either be determined individually or in an embodiment of the disclosure they can be determined simultaneously, e.g. using a chip or a bead based array technology. The concentrations of the biomarkers are then either interpreted independently, e.g., using an individual cut-off for each marker, or they are combined for interpretation.

As the skilled artisan will appreciate the step of correlating a marker level to a certain likelihood or risk can be performed and achieved in different ways. In some embodiments the determined concentrations of C-terminal proSP-B and of the one or more other marker(s) are mathematically combined and the combined value is correlated to the underlying diagnostic question. The one or more other marker value(s) may be combined with the determination of C-terminal proSP-B by any appropriate state of the art mathematical method.

The mathematical algorithm applied in the combination of markers may be a logistic function. The result of applying such mathematical algorithm or such logistical function may be, for example, a single value. Dependent on the underlying diagnostic question such value can easily be correlated to e.g., the risk of an individual for a damage in the broncheoalveolar compartment of the lung or to other intended diagnostic uses helpful in the assessment of patients with a damage in the broncheoalveolar compartment of the lung. In an illustrative way, such logistic function is obtained by a) classification of individuals into groups, e.g., into normals and individuals likely to have a damage in the broncheoalveolar compartment of the lung, b) identification of markers which differ significantly between these groups by univariate analysis, c) logistic regression analysis to assess the independent discriminative values of markers useful in assessing these different groups and d) construction of the logistic function to combine the independent discriminative values. In this type of analysis the markers are no longer independent but represent a marker combination.

In some embodiments the logistic function used for combining the values for C-terminal proSP-B and the value of at least one further marker is obtained by a) classification of individuals into the groups of normals and individuals likely to have a damage in the broncheoalveolar compartment of the lung, respectively, b) establishing the values for C-terminal proSP-B and the value of the at least one further marker c) performing logistic regression analysis and d) construction of the logistic function to combine the marker values for C-terminal proSP-B and the value of the at least one further marker.

A logistic function for correlating a marker combination to a disease may employ an algorithm developed and obtained by applying statistical methods. Appropriate statistical methods e.g. are Discriminant analysis (DA) (i.e., linear-, quadratic-, regularized-DA), Kernel Methods (i.e., SVM), Nonparametric Methods (i.e., k-Nearest-Neighbor Classifiers), PLS (Partial Least Squares), Tree-Based Methods (i.e., Logic Regression, CART, Random Forest Methods, Boosting/Bagging Methods), Generalized Linear Models (i.e., Logistic Regression), Principal Components based Methods (i.e., SIMCA), Generalized Additive Models, Fuzzy Logic based Methods, Neural Networks and Genetic Algorithms based Methods. The skilled artisan will have no problem in selecting an appropriate statistical method to evaluate a marker combination of the present disclosure and thereby to obtain an appropriate mathematical algorithm. In an embodiment the statistical method employed to obtain the mathematical algorithm used in the assessment of a damage in the broncheoalveolar compartment of the lung is selected from DA (i.e., Linear-, Quadratic-, Regularized Discriminant Analysis), Kernel Methods (i.e., SVM), Nonparametric Methods (i.e., k-Nearest-Neighbor Classifiers), PLS (Partial Least Squares), Tree-Based Methods (i.e., Logic Regression, CART, Random Forest Methods, Boosting Methods), or Generalized Linear Models (i.e., Logistic Regression). Details relating to these statistical methods are found in the following references: Ruczinski, I., et al., J. of Computational and Graphical Statistics 12 (2003) 475-511; Friedman, J. H., J. of the American Statistical Association 84 (1989) 165-175; Hastie, T., et al., The Elements of Statistical Learning, Springer Verlag (2001); Breiman, L., et al. Classification and regression trees, Wadsworth International Group, California (1984); Breiman, L., Machine Learning 45 (2001) 5-32; Pepe, M. S., The Statistical Evaluation of Medical Tests for Classification and Prediction, Oxford Statistical Science Series, 28, Oxford University Press (2003); and Duda, R. O., et al., Pattern Classification, John Wiley & Sons, Inc., 2nd ed. (2001).

It is an embodiment of the disclosure to use an optimized multivariate cut-off for the underlying combination of biological markers and to discriminate state A from state B, e.g., normals and individuals likely to have a damage in the broncheoalveolar compartment of the lung, patient having a damage in the broncheoalveolar compartment of the lung and being responsive to therapy and therapy failures, patients having a damage in the broncheoalveolar compartment of the lung without disease progression and patients having a damage in the broncheoalveolar compartment of the lung patients but showing disease progression, respectively.

The area under the receiver operator curve (=AUC) is an indicator of the performance or accuracy of a diagnostic procedure. Accuracy of a diagnostic method is best described by its receiver-operating characteristics (ROC) (see especially Zweig, M. H., and Campbell, G., Clin. Chem. 39 (1993) 561-577). The ROC graph is a plot of all of the sensitivity/specificity pairs resulting from continuously varying the decision thresh-hold over the entire range of data observed.

The clinical performance of a laboratory test depends on its diagnostic accuracy, or the ability to correctly classify subjects into clinically relevant subgroups. Diagnostic accuracy measures the test's ability to correctly distinguish two different conditions of the subjects investigated. Such conditions are for example, health and disease or disease progression versus no disease progression.

In each case, the ROC plot depicts the overlap between the two distributions by plotting the sensitivity versus 1-specificity for the complete range of decision thresholds. On the y-axis is sensitivity, or the true-positive fraction [defined as (number of true-positive test results)/(number of true-positive+number of false-negative test results)]. This has also been referred to as positivity in the presence of a disease or condition. It is calculated solely from the affected subgroup. On the x-axis is the false-positive fraction, or 1-specificity [defined as (number of false-positive results)/(number of true-negative+number of false-positive results)]. It is an index of specificity and is calculated entirely from the unaffected subgroup. Because the true- and false-positive fractions are calculated entirely separately, by using the test results from two different subgroups, the ROC plot is independent of the prevalence of disease in the sample. Each point on the ROC plot represents a sensitivity/1-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. (If the ROC plot falls completely below the 45° diagonal, this is easily remedied by reversing the criterion for “positivity” from “greater than” to “less than” or vice versa.) Qualitatively, the closer the plot is to the upper left corner, the higher the overall accuracy of the test.

One convenient goal to quantify the diagnostic accuracy of a laboratory test is to express its performance by a single number. The most common global measure is the area under the ROC plot (AUC). By convention, this area is always ≧0.5 (if it is not, one can reverse the decision rule to make it so). Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0).

The overall assay sensitivity will depend on the specificity required for practicing the method disclosed here. In certain exemplary settings a specificity of 75% may be sufficient and statistical methods and resulting algorithms can be based on this specificity requirement. In exemplary embodiment the method is used to assess individuals at risk for a damage in the broncheoalveolar compartment of the lung is based on a specificity of 80%, of 85%, or in some embodiments of 90% or of 95%.

In an embodiment the present disclosure relates to the use of C-terminal proSP-B as a marker molecule for obtaining an indication of a damage in the broncheoalveolar compartment of the lung in combination with one or more marker molecule(s) indicative for a damage in the broncheoalveolar compartment of the lung.

In one embodiment the present disclosure is directed to an in vitro method for obtaining an indication of a damage in the broncheoalveolar compartment of the lung by biochemical markers, comprising determining in a sample the concentration of C-terminal proSP-B and of one or more other marker(s), mathematically combining the determined concentration of C-terminal proSP-B and the concentration of the one or more other marker, respectively, wherein a increased combined value is indicative for the presence of a damage in the broncheoalveolar compartment of the lung.

Marker panels in one embodiment are combined within a single test device, e.g. on a chip or in an array format. A marker panel according to the present disclosure is in an embodiment determined using a bio-chip array (protein array) technique. An array is a collection of addressable individual markers. Such markers can be spatially addressable, such as arrays contained within microtiter plates or printed on planar surfaces where each marker is present at distinct X and Y coordinates. Alternatively, markers can be addressable based on tags, beads, nanoparticles, or physical properties. A bio-chip array can be prepared according to the methods known to the ordinarily skilled artisan (see for example, U.S. Pat. No. 5,807,522; Robinson, W. H., et al., Nat. Med. 8 (2002) 295-301; Robinson, W. H., et al., Arthritis Rheum. 46 (2002) 885-893). Array as used herein refers to any immunological assay with multiple addressable markers. A bio-chip array, also known to the skilled artisan as microarray, is a miniaturized form of an array.

The terms “chip”, “bio-chip”, “polymer-chip” or “protein-chip” are used interchangeably and refer to a collection of a large number of probes, markers or biochemical markers arranged on a shared substrate which could be a portion of a silicon wafer, a nylon strip, a plastic strip, or a glass slide.

An “array,” “macroarray” or “microarray” is an intentionally created collection of substances, such as molecules, markers, openings, microcoils, detectors and/or sensors, attached to or fabricated on a substrate or solid surface, such as glass, plastic, silicon chip or other material forming an array. The arrays can be used to measure the levels of large numbers, e.g., tens, thousands or millions, of reactions or combinations simultaneously. An array may also contain a small number of substances, e.g., one, a few or a dozen. The substances in the array can be identical or different from each other. The array can assume a variety of formats, e.g., libraries of soluble molecules, libraries of immobilized molecules, libraries of immobilized antibodies, libraries of compounds tethered to resin beads, silica chips, or other solid supports. The array could either be a macroarray or a microarray, depending on the size of the pads on the array. A macroarray generally contains pad sizes of about 300 microns or larger and can be easily imaged by gel and blot scanners. A microarray would generally contain pad sizes of less than 300 microns.

A “solid support” is insoluble, functionalized, polymeric material to which library members or reagents may be attached or covalently bound (often via a linker) to be immobilized or allowing them to be readily separated (by filtration, centrifugation, washing etc.) from excess reagents, soluble reaction by-products, or solvents.

The present disclosure also relates to a device for diagnosing a damage in the broncheoalveolar compartment of the lung, comprising a) an analyzing unit comprising a detection agent for determining the amount of C-terminal SP-B in a sample of a subject; and b) an evaluation unit comprising a data processor having tangibly embedded an algorithm for carrying out a comparison of the amount determined by the analyzing unit with a reference and which is capable of generating an output file containing a diagnosis established based on the said comparison.

The term “device” as used herein relates to a system of means comprising at least the aforementioned means operatively linked to each other as to allow the diagnosis. Exemplary means for determining the amount of the said C-terminal SP-B, e.g. the chips and arrays as specified herein above, and means for carrying out the comparison are disclosed above in connection with the methods of the disclosure. How to link the means in an operating manner will depend on the type of means included into the device. For example, where means for automatically determining the amount of C-terminal SP-B are applied, the data obtained by said automatically operating means can be processed by, e.g., a computer program in order to establish a diagnosis (i.e. identifying a subject suffering from damage in the broncheoalveolar compartment of the lung). In some exemplary embodiments, the means are comprised by a single device in such a case. Said device may accordingly include an analyzing unit for the measurement of the amount of the C-terminal SP-B in a sample and an evaluation unit for processing the resulting data for the diagnosis. Alternatively, where means such as test stripes are used for determining the amount of the C-terminal SP-B, the means for diagnosing may comprise control stripes or tables allocating the determined amount to an amount known to be accompanied with the presence of damage in the broncheoalveolar compartment of the lung or the absence of damage in the broncheoalveolar compartment of the lung. Exemplary means for detection are disclosed in connection with embodiments relating to the methods of the disclosure above. In such a case, the means are operatively linked in that the user of the system brings together the result of the determination of the amount and the diagnostic value thereof due to the instructions and interpretations given in a manual. The means may appear as separate devices in such an embodiment and are, for example, packaged together as a kit. The person skilled in the art will realize how to link the means without further inventive skills. Exemplary devices are those which can be applied without the particular knowledge of a specialized clinician, e.g., test stripes or electronic devices which merely require loading with a sample. The results may be given as output of parametric diagnostic raw data, for example, as absolute or relative amounts. It is to be understood that these data will need interpretation by the clinician. However, also envisaged are expert system devices wherein the output comprises processed diagnostic raw data the interpretation of which does not require a specialized clinician. Further exemplary devices may comprise the analyzing units/devices (e.g., biosensors, arrays, solid supports coupled to ligands specifically recognizing the polypeptides, Plasmon surface resonance devices, NMR spectro-meters, mass-spectrometers etc.) or evaluation units/devices referred to above in accordance with the methods of the disclosure.

Kit:

The present disclosure also provides a kit for performing the in vitro method according to the present disclosure comprising the reagents required to specifically determine the concentration of C-terminal proSP-B.

In one embodiment the present disclosure relates to a kit comprising at least two antibodies reactive with at least two non-overlapping epitopes comprised in the C-terminal proSP-B sequence of SEQ ID NO: 3. According to some embodiments, the at least two antibodies comprised in a kit are monoclonal antibodies.

Also disclosed is a kit according, comprising a first monoclonal antibody that reacts with an epitope comprised in the sequence stretch consisting of amino acids 285 to 294 of human proSP-B (SEQ ID NO: 4) and a second monoclonal antibody that reacts with an epitope comprised in the sequence stretch consisting of amino acids 323 to 334 of human proSP-B (SEQ ID NO: 5).

The present disclosure also provides a kit for performing the method according to the present disclosure comprising the reagents required to specifically determine the concentration of C-terminal proSP-B and optionally one or more marker protein of a damage in the broncheoalveolar compartment of the lung as described above.

In a further embodiment the present disclosure relates to an in vitro diagnostic medical device (IVD) for carrying out the method for obtaining an indication of a damage in the broncheoalveolar compartment of the lung according to the present disclosure.

In one embodiment the present disclosure relates to the use of the use of C-terminal proSP-B in obtaining an indication of a damage in the broncheoalveolar compartment of the lung

Experimental results for use of C-terminal proSP-B as an indicator of a damage in the broncheoalveolar compartment of the lung are shown in the example section.

The following examples, sequence listing, and figures are provided for the purpose of demonstrating various embodiments of the instant disclosure and aiding in an understanding of the present disclosure, the true scope of which is set forth in the appended claims. These examples are not intended to, and should not be understood as, limiting the scope or spirit of the instant disclosure in any way. It should also be understood that modifications can be made in the procedures set forth without departing from the spirit of the disclosure.

### ILLUSTRATIVE EMBODIMENTS

The following comprises a list of illustrative embodiments according to the instant disclosure which represent various embodiments of the instant disclosure. These illustrative embodiments are not intended to be exhaustive or limit the disclosure to the precise forms disclosed, but rather, these illustrative embodiments are provided to aide in further describing the instant disclosure so that others skilled in the art may utilize their teachings.
1. An in vitro method for diagnosing a damage in the broncheoalveolar compartment of the lung, the method comprising the steps of
   a) measuring the level of C-terminal proSP-B in a bodily fluid sample,
   b) comparing the level measured in (a) to a reference level of C-terminal proSP-B, wherein an increased level of C-terminal proSP-B is indicative of a damage in the broncheoalveolar compartment of the lung.
2. The method according to embodiment 1, wherein said measuring of C-terminal proSP-B is performed in a sandwich immuno assay employing at least two antibodies reactive with at least two non-overlapping epitopes comprised in the C-terminal proSP-B sequence (SEQ ID NO:2—positions 279 to 381).
3. The method according to embodiment 1 or 2, wherein said measuring of C-terminal proSP-B is performed in a sandwich immuno assay employing at least two antibodies reactive with at least two non-overlapping epitopes comprised in the C-terminal proSP-B sequence (SEQ ID NO:3—positions 285 to 334).
4. The method according to any of embodiments 1 to 3, wherein said detection is performed via a sandwich immuno assay.
5. The method according to any of embodiments 1 to 4, wherein said measuring is performed via a sandwich immuno assay employing two monoclonal antibodies.
6. The method according to any of embodiments 1 to 5, wherein a first monoclonal antibody is used that reacts with an epitope comprised in the sequence stretch consisting of amino acids 285 to 294 of human proSP-B (SEQ ID NO: 4).
7. The method according to any of embodiments 1 to 5, wherein a second monoclonal antibody is used that reacts with an epitope comprised in the sequence stretch consisting of amino acids 323 to 334 of human proSP-B (SEQ ID NO: 5).
8. The method according to embodiment 5, wherein a first monoclonal antibody that reacts with an epitope comprised in the sequence stretch consisting of amino acids 285 to 294 of human proSP-B (SEQ ID NO: 4) and a second monoclonal antibody that reacts with an epitope comprised in the sequence stretch consisting of amino acids 323 to 334 of human proSP-B (SEQ ID NO: 5) is used.
9. A kit comprising at least two antibodies reactive with at least two non-overlapping epitopes comprised in the C-terminal proSP-B sequence (SEQ ID NO:3—positions 285 to 334).
10. The kit according to embodiment 9 comprising two monoclonal antibodies.
11. The kit according to embodiment 10, comprising a first monoclonal antibody that reacts with an epitope comprised in the sequence stretch consisting of amino acids 285 to 294 of human proSP-B (SEQ ID NO: 4) and a second monoclonal antibody that reacts with an epitope comprised in the sequence stretch consisting of amino acids 323 to 334 of human proSP-B (SEQ ID NO: 5).

### EXAMPLES

### Example 1

### Assay for Measurement of proSP-B

### 1.1 Antibodies Used

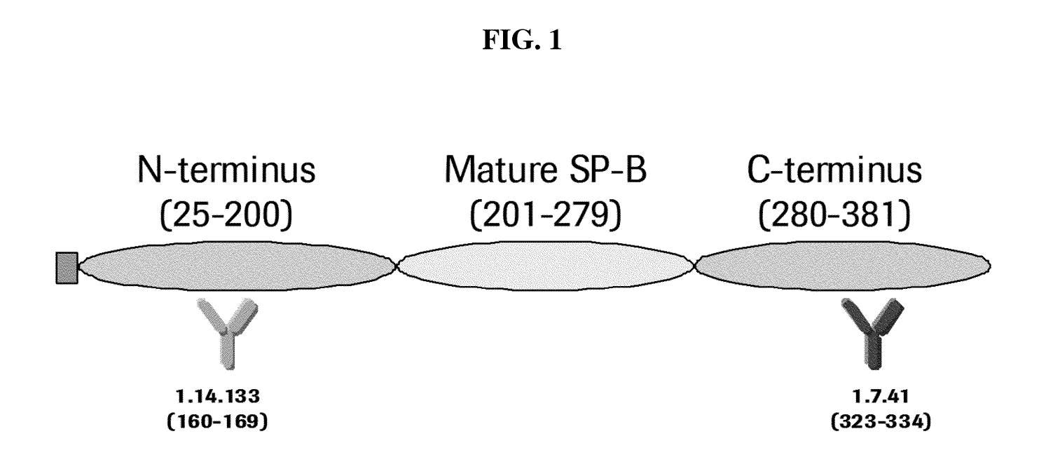

The pro SP-B assay uses a mouse monoclonal anti-proSP-B (N-terminus) antibody as a capture and a mouse monoclonal anti-proSP-B (C-terminus) antibody as a detection reagent. The assay principle is a sandwich format. In FIG. 1 this assay is schematically depicted. The antibody to the N-terminal pro-sequence (clone 1.14.133) binds to an epitope comprised in the peptide sequence ranging from amino acid 160 to 169 (SEQ ID NO: 6) of proSP-B. The antibody to the C-terminal pro-sequence (clone 1.7.41) binds to an epitope comprised in the peptide sequence ranging from amino acid 323 to 334 (SEQ ID NO: 5) of proSP-B. Detection is based on an electrochemiluminescence immunoassay (ECLIA), using a Tris(bipyridyl)-ruthenium(II) complex as label.

### 1.2 Assay Procedure

The biotinylated capture antibody (80 μl), the ruthenium-labeled detection antibody (80 μl), and sample or standard material (10 μl) are incubated in homogeneous phase for 9 min at 37° C. Concentrations in the stock solution were 1.7 μg/ml for the biotinylated capture antibody and 1.2 μg/ml for the ruthenylated detection antibody, respectively. After the first nine minutes 30 μl of Streptavidin-coated beads are added, and binding of the immune complexes formed to the microparticles takes place during a second 9-min incubation. After the second incubation, the reaction mixture is transferred into the measuring cell, where beads are captured to the electrode surface by a magnet. The measuring cell is washed to remove unbound label and filled with detection buffer containing Tris-propylamine. After applying voltage to the electrode, the emitted chemiluminescence light is detected by a photomultiplier. Results are determined via a 2-point calibration curve. The corresponding concentration for proSP-B is given in μg/ml.

### Example 2

### Assay for Measurement of C-Terminal proSP-B

### 2.1 Antibodies Used

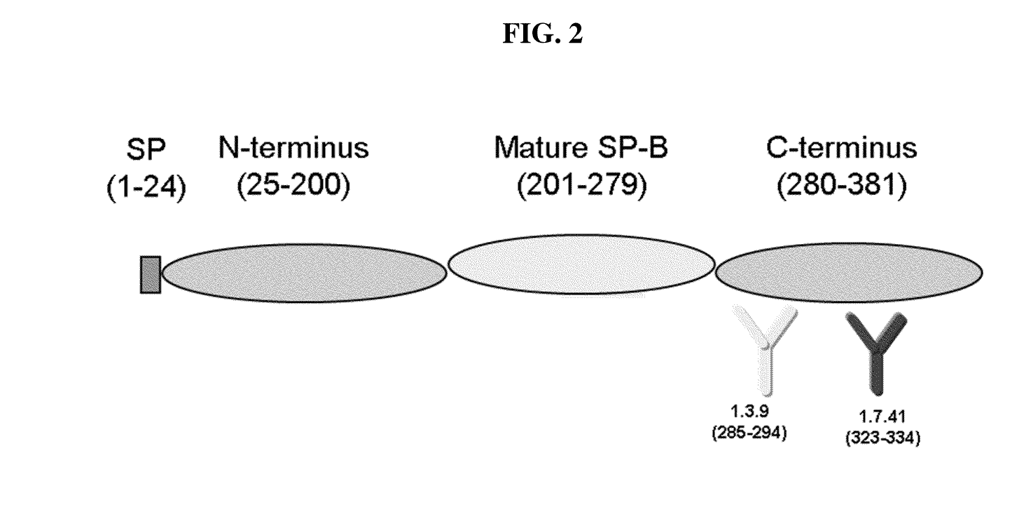

The pro SP-B assay uses a first mouse monoclonal anti-proSP-B (C-terminus) antibody as a capture and a second mouse monoclonal anti proSP-B (C-terminus) antibody as a detection reagent. The assay principle is a sandwich format. In FIG. 2 this assay is schematically depicted. The antibody to the first C-terminal pro-sequence (clone 1.7.41) binds to an epitope comprised in the peptide sequence ranging from amino acid 323 to 334 (SEQ ID NO: 5) of proSP-B. The antibody to the second C-terminal pro-sequence (clone 1.3.9) binds to an epitope comprised in the peptide sequence ranging from amino acid 285 to 294 (SEQ ID NO: 4) of proSP-B. Detection is based on an electrochemiluminescence immunoassay (ECLIA), using a Tris(bipyridyl)-ruthenium(II) complex as label. 72.2 Assay Procedure

The biotinylated capture antibody (MAB 1.7.41) (80 μL), the ruthenium-labeled detection antibody (MAB 1.3.9) (80 μl), and sample or standard material (10 μl) are incubated in homogeneous phase for 9 min at 37° C. Concentrations in the stock solution were 1.5 μg/ml for the biotinylated capture antibody and 1.0 μg/ml for the ruthenylated detection antibody, respectively. After the first nine minutes 30 μl of Streptavidin-coated beads are added, and binding of the immune complexes formed to the microparticles takes place during a second 9-min incubation. After the second incubation, the reaction mixture is transferred into the measuring cell, where beads are captured to the electrode surface by a magnet. The measuring cell is washed to remove unbound label and filled with detection buffer containing Tris-propylamine. After applying voltage to the electrode, the emitted chemiluminescence light is detected by a photomultiplier. Results are determined via a 2-point calibration curve. The corresponding concentration for C-terminal proSP-B is given in μg/ml.

### Example 3

### Investigation of Clinical Samples

### 3.1 Groups of Clinical Specimen Investigated

The groups X of clinical sample investigated are given in Table 1:

TABLE 1
Samples investigated

| | number of |
|---|---|
| | samples investigated |
| non-smoker | 20 |
| smoker | 25 |
| Occup. risk | 38 |
| COPD | 27 |
| Pneumonia | 28 |
| Pneumoconiosis | 62 |
| Sarcoidosis | 17 |
| Asthma | 25 |
| NSCLC | 52 |

### 3.2 Relative Increase in the Values Measured

For both assays, proSP-B and C-terminal proSP-B, respectively, the values measured were normalized to the reference values established on the basis of samples obtained from 20 healthy non-smoking individuals. The median values for each group X have been divided by the median value of the two markers as established in the reference group. The relative increase in the values for proSP-B as compared to C-terminal proSP-B is given in Table 2.

TABLE 2
Median values for each group X and relative increases for both
the markers proSP-B and C-terminal proSP-B, respectively.

| | | | | ratio of group X | | |
|---|---|---|---|---|---|---|
| | Median | | | to healthy nonsmokers | | |
| | proSP- | C-proSP- | | proSP- | C-proSP- | |
| group X | B | B | Δ¹ | B | B | Δ |
| nonsmoker | 0.028 | 0.0455 | 0.018 | 1.00 | 1.00 | 1.00 |
| smoker | 0.092 | 0.192 | 0.100 | 3.29 | 4.22 | 5.56 |
| Occup. risk | 0.0435 | 0.085 | 0.042 | 1.55 | 1.87 | 2.31 |
| COPD | 0.115 | 0.252 | 0.137 | 4.11 | 5.54 | 7.61 |
| Pneumonia | 0.049 | 0.097 | 0.048 | 1.75 | 2.13 | 2.67 |
| Pneumo- | 0.1035 | 0.228 | 0.125 | 3.70 | 5.01 | 6.92 |
| coniosis | | | | | | |
| Sarcoidosis | 0.07 | 0.121 | 0.051 | 2.50 | 2.66 | 2.83 |
| Asthma | 0.03 | 0.047 | 0.017 | 1.07 | 1.03 | 0.94 |
| NSCLC | 0.102 | 0.2355 | 0.134 | 3.64 | 5.18 | 7.42 |

Δ¹ = ((C-proSP-B)-(proSP-B))

### 2.3 Interpretation of Data

In the group of patients with asthma, no significant increase in either proSP-B or C-terminal proSP-B is observed. This is in line with expectations, since asthma is not known to cause a damage in the broncheoalveolar compartment of the lung.

In all those groups suspected to suffer from a damage to the broncheoalveolar compartment of the lung there is an increase in both proSP-B and C-terminal proSP-B. Surprisingly the relative increase is always higher for C-terminal proSP-B. We thus conclude that surprisingly C-terminal proSP-B represents a superior marker as compared to proSP-B for obtaining an indication of a damage to the broncheoalveolar compartment of the lung. This view is further corroborated if polypeptides comprising the N-terminal 200 amino acids are omitted from the analysis (columns “Median/Δ” and “ratio of group X to healthy non-smokers/Δ”): the assay of Example 2 determines the amount of all polypeptides comprising C-terminal amino acids 285-334 of SEQ ID NO: 1 in a sample, including sequences comprising the N-terminal 200 amino acids. The assay of Example 1 specifically determines the amount of polypeptides comprising the N-terminal 200 amino acids, excluding polypeptides lacking said N-terminal amino acids. Thus, the concentration of polypeptides comprising said C-terminal but not comprising said N-terminal amino acids can be determined by subtracting the value obtained in the assay of Example 1 from the value obtained in the assay of Example 2. It is clear from the values in the rightmost column of Table 2 that the sensitivity of the determination is even higher when only polypeptides comprising C-terminal proSP-B but not N-terminal proSP-B are taken into account.

### Example 4

### Investigation of Clinical Samples from Risk Groups

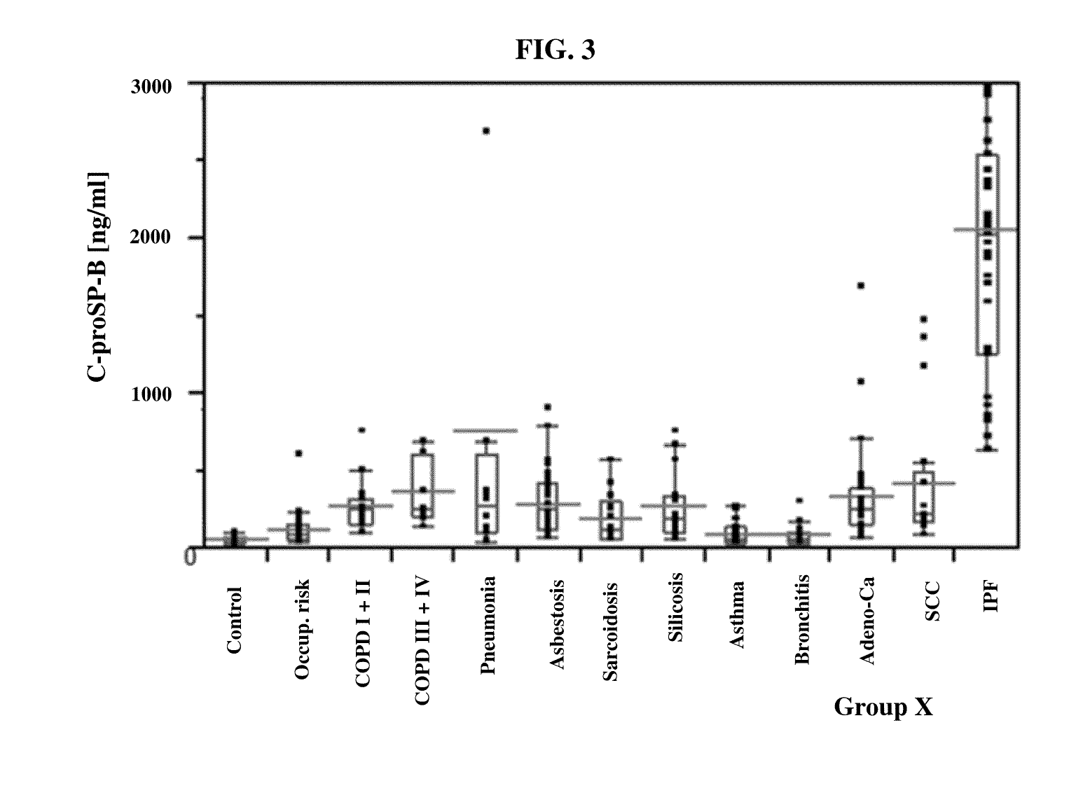

A total number of 304 serum samples from subjects from various risk groups for suffering from damage in the broncheoalveolar compartment of the lung or from control subjects were analysed with the assay for measurement of C-terminal proSP-B according to Example 2. Statistical evaluations are shown in Table 3 and FIG. 3. The concentration for C-terminal proSP-B is given in ng/ml.

TABLE 3
Statistical analysis of values for various
groups X and C-terminal proSP-B.

| | Mini- | | | | | | Maxi- |
|---|---|---|---|---|---|---|---|
| group X | mum | 10% | 25% | Median | 75% | 90% | mum |
| control_non- | 24 | 24.1 | 31.5 | 45.5 | 63 | 79.1 | 108 |
| smoker | | | | | | | |
| n = 20 | | | | | | | |
| control_oc- | 32 | 40.9 | 52 | 85 | 145.5 | 191.3 | 595 |
| cup.risk | | | | | | | |
| n = 39 | | | | | | | |
| COPD I + II | 100 | 100.9 | 152.5 | 245 | 314 | 519.8 | 752 |
| n = 18 | | | | | | | |
| COPD | 137 | 137 | 207 | 252 | 608 | 690 | 690 |
| III + IV | | | | | | | |
| n = 9 | | | | | | | |
| Pneumonia | 35 | 38.9 | 97 | 260.5 | 604.5 | 3553.9 | 3925 |
| n = 12 | | | | | | | |
| Asbestosis | 59 | 76.7 | 113.5 | 254.5 | 409.25 | 503.2 | 898 |
| n = 46 | | | | | | | |
| Sarcoidosis | 42 | 43.6 | 50 | 121 | 298 | 451.4 | 565 |
| n = 18 | | | | | | | |
| Silicosis | 53 | 62.1 | 96.25 | 178 | 328.5 | 697.9 | 756 |
| n = 16 | | | | | | | |
| Asthma | 22 | 27.6 | 37 | 47 | 139.5 | 207.8 | 267 |
| n = 25 | | | | | | | |
| Bronchitis | 21 | 24.5 | 33.75 | 55.5 | 107.5 | 210.8 | 299 |
| n = 16 | | | | | | | |
| Adeno-Ca | 75 | 107.8 | 151 | 243 | 380 | 775.2 | 1689 |
| n = 29 | | | | | | | |
| SCC | 89 | 114.6 | 163 | 212 | 489.5 | 1368 | 1472 |
| n = 17 | | | | | | | |
| IPF | 627.8 | 842 | 1253 | 2012 | 2528 | 2967 | 6676 |
| n = 39 | | | | | | | |

All references cited in this specification are herewith incorporated by reference with respect to their entire disclosure content and the disclosure content specifically mentioned in this specification.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within the known or customary practice in the art to which this disclosure pertains.

What is claimed is:

1. An in vitro method for diagnosing a damage in a broncheoalveolar compartment of a lung of a patient, comprising:
determining a level of C-terminal proSP-B in a bodily fluid sample obtained from the patient;
comparing the level of C-terminal proSP-B in the sample determined in said step of determining with a C-terminal proSP-B reference level;
providing a diagnosis of damage in the broncheoalveolar compartment of the lung of the patient if the concentration of C-terminal proSP-B in the sample determined in said step of determining is greater than the C-terminal proSP-B reference level.

2. The method according to claim 1, wherein said step of determining comprises an immunoassay procedure.

3. The method according to claim 2, wherein the immunoassay procedure comprises a sandwich assay format.

4. The method according to claim 2, wherein the immunoassay procedure comprises a competitive assay format.

5. The method according to claim 2, wherein the immunoassay procedure comprises an electrochemiluminescence immunoassay (ECLIA).

6. The method according to claim 1, wherein the C-terminal proSP-B reference level has a specificity of 95%.

7. The method according to claim 1, wherein said step of determining further comprises the steps of:
contacting a portion of the sample obtained from the patient with a detection antibody having specific binding affinity for C-terminal proSP-B, thereby forming a complex between the detection antibody and C-terminal proSP-B, the detection antibody having a detectable label;
separating the complex formed in said step of contacting from detection antibody not comprising the complex; and
quantifying a signal from the detectable label of the detection antibody comprising the complex formed in said step of contacting, the signal being proportional to an amount of C-terminal proSP-B in the sample obtained from the patient, whereby an amount of C-terminal proSP-B in the sample obtained from the patient is calculated.

8. The method of claim 7 further comprising the step of contacting the portion of the sample from the subject with a capture antibody, the capture antibody having specific binding affinity for an epitope of C-terminal proSP-B not bound by the detection antibody, thereby forming a complex between the capture antibody and C-terminal proSP-B, the capture antibody coupled to one of streptavidin and biotin, said step of contacting the portion of the sample with the capture antibody occurring prior to said steps of separating and quantifying,
wherein upon said steps of contacting the portion of the sample with the detection antibody and contacting the portion of the sample with the capture antibody, a complex between the detection antibody, C-terminal proSP-B and the capture antibody is thereby formed.

9. The method of claim 7, wherein the detectable label is a ruthenium complex.

10. The method of claim 7, wherein said step of quantifying a signal comprises use of a computing device.

11. The method of claim 7, wherein said step of contacting and said step of separating comprise use of a medical device.

12. The method of claim 8, wherein the capture antibody and detection antibody comprise specific binding affinity for separate, non-overlapping epitopes of C-terminal proSP-B amino acid positions 279 to 381 (SEQ ID NO.2).

13. The method of claim 8, wherein the capture antibody and detection antibody comprise specific binding affinity for separate, non-overlapping epitopes of C-terminal proSP-B amino acid positions 285 to 334 (SEQ ID NO.3).

14. The method of claim 8, wherein the capture antibody and detection antibody comprise monoclonal antibodies.

15. The method of claim 14, wherein one of the capture antibody and detection antibody comprise specific binding affinity for an epitope comprised of amino acids within amino acids 285 to 294 (SEQ ID NO.4) of human proSP-B and the other of the capture antibody and detection antibody comprise specific binding affinity for an epitope comprised of amino acids within amino acids 323 to 334 (SEQ ID NO.5).

16. A kit for performing the method of claim 1 comprising:
a capture antibody; and
a detection antibody,
the capture antibody and the detection antibody being reactive with at least two non-overlapping epitopes comprised in the C-terminal proSP-B sequence (SEQ ID NO:3—positions 285 to 334), the capture antibody bound to or configured for binding to a solid phase support and the detection antibody having a detectable label bound thereto.

17. The kit of claim 16, wherein the capture antibody and the detection antibody are monoclonal antibodies.

18. The kit of claim 17, wherein one of the capture antibody and detection antibody comprises specific binding affinity for an epitope comprised of amino acids within amino acids 285 to 294 (SEQ ID NO.4) of human proSP-B and the other of the capture antibody and detection antibody comprises specific binding affinity for an epitope comprised of amino acids within amino acids 323 to 334 (SEQ ID NO.5).

19. The kit of claim 16, wherein the solid phase support is a bead coupled to one of streptavidin and biotin and the capture antibody is coupled to the other of streptavidin and biotin, and wherein the detectable label of the detection antibody comprises ruthenium.
